(19) United States
(12) Patent Application Publication
(10) Pub. No.: US 2020/0281727 A1
(43) Pub. Date: Sep. 10, 2020

(54) SELECTIVE NANO SURFACE MODULATION OF MEDICAL DEVICES

(71) Applicant: PrinterPrezz, Inc., Fremont, CA (US)

(72) Inventors: Alan B.C. Dang, Fremont, CA (US); Alexis B.C. Dang, Fremont, CA (US); Shrinivas Shetty, Fremont, CA (US); Pawan Kapur, Fremont, CA (US)

(21) Appl. No.: 16/789,419

(22) Filed: Feb. 12, 2020

Related U.S. Application Data
(60) Provisional application No. 62/804,283, filed on Feb. 12, 2019.
(60) Provisional application No. 62/804,510, filed on Feb. 12, 2019.
(60) Provisional application No. 62/805,274, filed on Feb. 13, 2019.
(60) Provisional application No. 62/805,279, filed on Feb. 13, 2019.

(51) Int. Cl. A61F 2/30

(52) U.S. Cl. CPC A61F 2/3094; A61F 2/30767; A61F 2002/30925; A61F 2002/30968; A61F 2002/30321; A61F 2002/30962

(57) ABSTRACT

Fabrication methods and structures for medical devices having selective surface modulation are provided.

### CROSS-REFERENCE TO RELATED APPLICATIONS

This application claims the benefit of U.S. Provisional Patent Application Ser. Nos. 62/804,283 filed Feb. 12, 2019, App. No. 62/804,510 filed Feb. 12, 2019, App. No. 62/805,274 filed Feb. 13, 2019, and App. No. 62/805,279 filed Feb. 13, 2019 all of which are hereby incorporated by reference in their entirety.

### FIELD

The present disclosure relates in general to the fields of medical devices, and more particularly to selective surface modulation of medical devices.

### BACKGROUND

Medical devices (e.g. implants, surgical instruments, surgical tools) may be manufactured through any number of primary methods. Examples of primary manufacturing methods include casting, forging, milling, sintering, and 3D printing (additive manufacturing). The optimal method for manufacturing a particular medical device may differ based upon the intended design specifications, cost concerns, production time, or materials such as plastics, metals, or ceramics.

Clinically, there are applications where rough surfaces are desired (e.g., to increase bony integration or increase friction between two structures) and applications where smoother surfaces are desired (e.g., to minimize tissue irritation/inflammation or minimize friction between two structures).

In order to provide different levels of friction across portions of a medical device, computer assisted design files or equivalent blueprints may introduce design features capable of being reproduced through primary manufacturing. For example, a sawtooth ridge pattern may be designed onto a surface intended to have increased friction/roughness relative to other portions of the implant such as in a spinal implant, a cylindrical shaft may be designed onto a surface intended to have increased friction/roughness relative to other portions of the implant such as an implant for small joint hemiarthroplasty, a keel may be used in a glenoid replacement to enhance ingrowth into the scapula, or sharp corners may be designed such as in a bone rasp on bone file. For example, a plate used for bone fixation may feature scalloped indentations or recessions on the portion of the plate in contact with the bone to increase friction along the bony surface and decrease the total surface area of an implant on the bone.

It is generally understood that primary design features to introduce surface roughness include ridges having a minimum feature size in the range of 0.25 mm to 1.5 mm for ridges (see U.S. Pat. Pub. No. US20020143400A1; FIGS. 1-3). Larger feature designs such as a keel may be used as well (see U.S. Pat. Pub. No. US20050149192A1; FIGS. 1-18 and U.S. Pat. No. 8,080,063B2; FIGS. 2, 4-5). And it is generally understood that primary design features to introduce surface roughness may include shafts with a minimum diameter size in the range of 3 mm to 4 mm such as that in a distal phalanx (see U.S. Pat. Pub. No. US20070185584A1; FIGS. 1-3). And it is generally understood that primary design features to introduce surface roughness include features such as indentations having a minimum feature size in the range of 2 mm to 4 mm (see U.S. Pat. Pub. No. US20110137314A1; FIG. 1).

These ridges, keels, and scallops exist at a large millimeter scale. The surface of medical implants, including the surface of a ridge and surface of a keel, is generally consistent across the surface of the implant in terms of generally consistent roughness or consistent smoothness.

Primary design features in the design of the medical device itself are limited by the resolution of the manufacturing technique. For example, in a medical device manufactured by a computerized numerical control mill, the type of cutting bit and precision/resolution of the positioning of the cutting bit defines the smoothness or roughness surface characteristic. In powder bed metal 3D printing, the precision of a laser in terms of spot size and ability to position a laser across three dimensional space as well as the average grain size of the powdered metal reflects the precision achievable through primary manufacturing.

And despite the increased functionality provided by 3D printing, there are still several limitations to these 3D-printed devices. Limitations of selective metal powder sintering (laser/electron-beam spot size) creates a lower limit on lattice struct thickness on the order of 0.4 mm. Due to imperfections of the source powder (particulate size variation, contaminants, etc.) and residual stresses resulting from the selective sintering processes, operating at the limits of the 3D printing technology result in design-construction discrepancies including strut lattice mismatch, incomplete struts, and inconsistent mechanical properties caused by stochastic point defects/contaminants. Additionally, while it is possible to modulate the wall/strut thickness of 3D-printed parts, methods to modulate surface roughness are limited and the resulting parts have a lower limit on the order of 4.5 Ra. Reducing surface roughness further requires post-processing procedures such as sanding and/or polishing.

These shortcomings limit the functionality of 3D-printed implants, particularly when leveraging its ability to manufacture complex internal lattice structures. When the lattice is sparse and the struts are thin, the residual stress in the part may cause the lattice to warp, causing dislocation and misalignment as the lattice is expanded, resulting in part failure. On the opposite end of the spectrum, when the lattice is dense, access to the interior surface for smoothing/polishing becomes a challenge, resulting in increased surface area for bacterial growth and reduced patient outcomes.

One method of post processing to enhance smoothness is the subtractive process of polishing with abrasive materials. Exemplary abrasive materials include aluminum oxide, corundum, diamond, emery, cubic boron nitride, garnet, zirconia, silicon carbide, silica, iron oxide. Polishing using these abrasive materials allows some selectivity of surface finish based upon where the mechanical polishing wheel/brush/bur is being applied. Although this can provide some selective enhancement of surface smoothness, the precision is still limited by the primary resolution of the tool. Additionally, this can introduce contaminants to the finished product. This type of post processing is well known in the art and is not novel. “Mirror polishing” has been described for selective finishing of bone plates (see U.S. Pat. Pub. No. US20060025772A1).

Another method of post processing to enhance smoothness or to enhance roughness is shot blasting. Shot blasting involves shooting a high-pressure stream of abrasive material (also known as shots or blasting media) against the surface of a metal part. Depending on the application, the shots may be propelled by a pressured fluid (like compressed air) or a centrifugal wheel (also known as wheel blasting). The shape, size and density of the shots will determine the final results. Types of metal abrasives used in shot blasting include steel grit, copper shots, and aluminum pellets. Other methods of shot blasting use silica sand, glass beads, synthetic materials like sodium bicarbonate (baking soda), and even agricultural materials like crushed walnut kernels. Grit blasting is one form of shot blasting using irregular coarse shot. This can be used to impart a roughened surface texture. Glass bead blasting is another form of shot blasting in which glass beads are used to achieve a smoother surface. This type of post processing is well known in the art and is not novel.

A disadvantage of shot blasting or wheel blasting is that the surface treated is broad and cannot be specified to individual portions of an implant other than direction of the pressurized shot. This can be a particular challenge in a latticed implant.

### BRIEF SUMMARY OF THE INVENTION

Therefore, a need has arisen for medical devices having selective surface modulation. In accordance with the disclosed subject matter, medical devices having selective surface modulation are provided which substantially eliminate or reduce disadvantages and deficiencies associated with previously developed medical devices having selective surface modulation.

According to one aspect of the disclosed subject matter, a method for forming a medical device is provided. A three dimensional medical structure having x, y, and z structural geometry in a three dimensional x, y, and z Cartesian coordinate system is formed. The surface roughness of a portion of the three dimensional medical structure is selectively modulated to have surface roughness features less than 0.1 mm.

According to another aspect of the disclosed subject matter, a medical device is provided. A three dimensional medical structural component has x, y, and z structural geometry in a three dimensional x, y, and z Cartesian coordinate system. The three dimensional medical structural component made of at least a structural material and a nano-modulated surface roughness on a portion of the device that includes at least one but not all contact surfaces. A nano-modulated surface roughness is controlled via the addition, coating, deposition, or deformation of structural or non-structural material in valleys of the contact surface. A nano-modulated surface roughness is controlled via the removal, coating, deposition, or deformation of material in the peaks of the contact surface. The surface roughness of a portion of the three dimensional medical structure to has surface roughness features less than 0.1 mm.

These and other aspects of the disclosed subject matter, as well as additional novel features, will be apparent from the description provided herein. The intent of this summary is not to be a comprehensive description of the claimed subject matter, but rather to provide a short overview of some of the subject matter's functionality. Other systems, methods, features and advantages here provided will become apparent to one with skill in the art upon examination of the following FIGUREs and detailed description. It is intended that all such additional systems, methods, features and advantages that are included within this description, be within the scope of any claims.

### DETAILED DESCRIPTION

The following description is not to be taken in a limiting sense, but is made for the purpose of describing the general principles of the present disclosure. The scope of the present disclosure should be determined with reference to the claims. Exemplary embodiments of the present disclosure may be illustrated in the drawings, like numbers being used to refer to like and corresponding parts of the various drawings. The dimensions of drawings provided are not shown to scale.

The scope of the invention may be advantageously directed toward medical devices that may be implanted in the body or be in contact with the body where selective surface roughness is desired, for example for reduction of neural, cartilage, and tendon inflammation and friction as well as to maximize tendon gliding.

The term “selective” is intended to describe a design in which portions of a medical device are intentionally designed with a different level of surface roughness than other geographic portions of the medical device.

The term “selective” can also be used to describe a surface characteristic that is anisotropic or directional in design and function. For example, to guide the path of growth, promote adhesion, or prevent adhesion of cells and tissues.

The term “surface modulation” is used to describe any method of manufacturing that alters or modifies the surface finish (e.g., surface texture) of a material. A surface finish is synonymous with surface quality and reflects the roughness or smoothness of surface. The surface roughness can be quantified by deviations in the direction of the normal vector of a real surface from its ideal form.

The term “nano” in the context of “selective nano surface modulation” is intended to reflect surface modulation (e.g., surface roughness correction, roughness smoothing, or roughness enhancing) at the level of less than 0.1mm (<0.1mm) in feature (i.e., surface roughness feature) size.

The terms “peaks” and “valleys” are used to describe proximate highest and lowest surface features to convey surface roughness.

The scope of this invention may be additionally advantageously directed toward the application of “selective nano surface modulation.”

The terminology used herein is for the purpose of describing particular embodiments only and is not intended to be limiting of the claims. As used in the description of the embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

It is understood that terms post-processing or secondary manufacturing are used contextually to separate it from primary manufacturing but is not intended to convey a sequential or serial process. While some embodiments of the invention do apply post-processing after the complete manufacturing of an initial medical device through a primary manufacturing method, other embodiments allow for simultaneous post-processing during the process of building (i.e. 3D printing, where secondary manufacturing is performed between layers of primary manufacturing). Other embodiments allow for a pre-processing of secondary manufacturing such as in modification of materials such as powders or filaments used to supply primary manufacturing tools. Thus, post processing should be understood as a method or system of secondary manufacturing but that secondary manufacturing includes real-time processing and pre-processing. Exemplary material selection for primary manufacturing includes metals such as titanium as well as plastics such as polyetheretherketone (PEEK) and polyetherketoneketone (PEKK). Primary manufacturing may include structural materials as well as non-structural materials.

The current invention relates to method, system, and design features introduced through a secondary manufacturing process. Secondary manufacturing can be used almost interchangeably with post-processing. As described herein, secondary manufacturing is a method, system, and design that modifies the smoothness or roughness of the surface of a medical device, for example a medical implant, at a smaller feature level, i.e. less than 0.1 mm (<0.1 mm) in feature size.

The solutions of the present application may be particularly advantageous to selectively modify the surface of portions of a medical device. Advantageously, the selective nano surface modulation of medical devices solutions provided may, for example, minimize neural, cartilage, and tendon irritation and inflammation and maximize tendon gliding.

The medical device solutions disclosed provide medical devices having selective surface modification, for example selective smoothness or roughness, on a portion of the medical device such that the selectively modified surface of the medical device is different than the surface of non-modified surface portions of the medical device. The solutions of the provided herein may be particularly advantageous to selectively smooth surface portions of a medical device, or correct surface roughness, in the range of less than 0.1 mm (<0.1 mm).

For example, a medical device having saw tooth blade type surface features such that device structure tooth peaks and between teeth valleys form the teeth of the saw tooth. The medical device solutions disclosed herein provide structures and methods for modified saw tooth teeth via selective surface modification, for example: distorted or bent saw tooth teeth (i.e., deformation); teeth tip removal using removable wax masking of valleys and tip material removal. Decreasing teeth height through modification may also be performed using filling, deposition, or selective coating material (e.g., using masking of peaks such as removable wax masking of peaks) in teeth valleys.

The inventors of the present disclosure have advantageously determined that selective nano surface modulation may be used during the secondary manufacturing of medical devices to minimize, for example, neural, cartilage, and tendon friction, irritation, or inflammation on areas that are in temporary or prolonged contact with neural structures such as dura, nerve roots, nerve rootlets, or peripheral nerves and articular cartilage or other joint contacting structures such as subchondral bone in areas of cartilage damage, synovial tissue, plica tissue, labral tissue, or areas in close proximity to neural structures and joint surfaces or tendons or other tendon contacting structures such as muscle, fascia, a tendon sheath, or in close proximity to joint surfaces.

A medical device that has been enhanced with selective nano surface modulation comprises various elements. The first element is a primary manufactured medical device. This device can be manufactured through traditional subtractive or additive 3D printing using any method known to the art.

The contact surfaces of the medical device represent the second element of the disclosure. In a cube, there are six, discrete large contact surfaces. The linear edges and pointed corners of the cube also represent other contact surfaces. In a sphere, there are numerous contact surfaces at a plane tangent to every point on the surface of the sphere.

The third element of this invention relates to a medical device with selective surface quality. A selective surface quality is a surface quality where at least one of the contact surfaces is intended to have a different surface finish than other contact surfaces on the medical device. In some instances there may be two surfaces finishes on a medical device while in others, there may be more than two desired surface finishes on the medical device. The upper limit would represent the total number of contact surfaces minus one (i.e., two contact surfaces have identical desired surface qualities with every other contact surface having a different desired surface quality).

The fourth element of the invention is that at least one or more of the contact surfaces will be in close proximity or direct contact with neural, cartilaginous structures, or tendons either temporarily or for a prolonged period. Examples of direct contact with neural structures include contact of a medical device contact surface with a nerve, a nerve root, or dural tissue. Examples of direct contact with cartilaginous structures include contact of a medical device contact surface with an articular surface such as a hemiarthroplasty prosthetic device. Examples of direct contact with tendons structures include contact of a medical device contact surface with extensor or flexor tendons of crossing the wrist/forearm. Examples of anatomic structures in close proximity to neural structures include perineural or epidural adipose tissue (fat), perineural or epidural vessels, the ligamentum flavum or posterior longitudinal ligaments of the spine, bones, muscles, ligaments, tendons, and other connective tissue. Examples of anatomic structures in close proximity to cartilaginous structures include labral tissue, synovial or capsular tissue, intra-articular ligaments such as the anterior cruciate or posterior cruciate ligaments, intra-articular vessels such as the middle geniculate as well other bones, muscles, ligaments, tendons, and other connective tissue. Examples of anatomic structures in close proximity to tendinous structures include muscle, fascial tissue, skin, bones, ligaments, nerves, and other connective tissue.

A contact surface in close proximity to neural, cartilage, joint surface structures, or tendons is defined as a contact surface that at any time, during the course of dynamic physiologic movement, the course of intrinsic implant movement, the course of a surgery, the course of a medical procedure, or the course of powering on an electronic medical device, or otherwise activating a medical device with mechanical or chemical energy storage, contacts, distorts, displaces, deforms or otherwise generates a mass effect or physical movement or change in pressure upon the underlying neural or cartilaginous tissues or tendons through any number of cascading contact.

In one embodiment of the invention, the method of nano surface modulation is low plasticity burnishing (LPB). In low plasticity burnishing (LPB), pressure applied to a surface can cause plastic deformation to occur in the surface of the material directly in contact with the LPB tool, relying on the bulk of the material to constrain the deformed area, leaving the deformed zone in compression. Repeated methodologic application of the pressure across the contact surface is used to homogenize the surface. Typical surface roughness after extended LPB for a titanium alloy Ti6Al4V-ELI medical device can reach less than 0.1 microns (or 0.0001 mm). Selectivity may be enhanced by physically limiting the portions of the medical device (e.g., contact surfaces) that the LPB tool is in contact with. As LPB relies on plastic deformation of a surface as opposed to a mechanically abrasive mechanism additional contaminants may be reduced.

In another embodiment, a medical device may undergo an electropolishing in a dry or wet process. Electropolishing relies on an electrochemical process where a reduction reaction is performed on the surface of a material resulting in a portion of the medical device being oxidized and then being dissolved into the electrolyte. As protruding parts of a surface profile dissolve faster than the recessed portions, the surface is leveled. Electropolishing has an advantage of being effective with irregularly shaped contact surfaces and fragile surfaces that may not withstand mechanical deformation. Typical surface roughness after electropolishing of a titanium alloy Ti6Al4V-ELI medical device can reach less than 0.0005 mm. Selectivity can be enhanced by insulating or reducing the electrical conductivity of a specific contact surface or altering the temperature or thermal characteristics of a contact surface through any method known to the art. Removal of material using an electropolishing method results in small amounts of material (as small as the angstrom level) being removed and dissolved. This reduces contaminants as there are no bulk abrasive materials embedded on the surface of the medical device after electropolishing. In some embodiments of the invention, the electrolyte is either fully miscible with a biocompatible cleaning media allowing all contaminants to be removed. In other embodiments of the invention, the electrolyte can be placed under “sink conditions” in which a sufficient volume of a biocompatible cleaning media is used in which all of the active electrolyte and any excipient material is washed away. In other embodiments of the invention, the electrolyte is a “dry” format in which electrically conductive free solid bodies charged with a negative electrical charge in a gaseous environment wherein any amount of electrolyte fluid may be retained by particles below a saturation level are used for electropolishing, thereby avoiding any free electrolyte fluid on the surface. It is understood that wet etching and electropolishing are used synonymously,

In another embodiment, a medical device may undergo an optical peening method. In this method, a high energy laser may be used to generate an expanding plasma which applies pressure pulses of about one million pounds per square inch, sending shock waves through the specimen generating a compressive force to generate a similar characteristic as that achieved by low plasticity burnishing. Selectivity may be enhanced by applying optical masks, paints, or resins to specific contact surfaces. Optical peening allows removal of material in very small amounts (as small as angstrom level) to reduce contaminants.

In another embodiment, shot peening or ultrasonic shot peening is used, but with the impacting material or materials resulting in a surface finish <0.1 mm. Example parameters for shot peening include an initial high-intensity, cut-wire peening (SSCW 0.4/d=400 um/Almen intensity 0.012″A/coverage=200%) followed by a low-intensity cleaning process using glass blasting medium to remove contaminants from the cut-wire (GP 60/d=60-150 um/Almen intensity 0.006″N/coverage=125%). An example set of parameters for ultrasonic shot peening would be to use ceramic beads (d=1.0-1.2 mm, hardness=1200 HV) and carrying out the process by a closed acoustic block where the beads are placed under an ultrasonic frequency (amplitude=80 peening intensity=F20.12A) for 120 seconds, forming a ‘bead gas’ with intended coverage>125%). For titanium alloy Ti6Al4V-ELI implants, the use of bead peening generally achieves a surface Ra of less than 0.002 mm. Selectivity may be enhanced by physically limiting the portions of the medical device (e.g., contact surfaces) that the ultrasonic bead peening chamber is in contact with, for example a barrel/chamber that mechanically only exposes specific portions of the medical device/implant to bead peening. The use of peening relies on plastic deformation of a material rather than abrasion, thereby reducing contaminants.

In another embodiment a dry etching technique may be used in conjunction with photolithographic techniques. This is done by exposing a contact surface to a bombardment of ions. In some embodiments this is done using a plasma of reactive gases (e.g. fluorocarbons, oxygen, chlorine, boron trichloride, or sulfur hexafluoride). In some embodiments this is done using physical sputtering using inert gases such nitrogen, argon, or helium. In some embodiments this is done in a vacuum and in other embodiments it is with the addition of a gas or combination of gases such as nitrogen, argon, or helium. This dislodges portions of the material from the exposed surface that chemically react with gas plasma. Use of a plasma gas is advantageous in allowing for small amounts of material to be removed (as small as an angstrom level) additionally minimizing contaminants.

For example in a medical orthopedic implant, orthopedic implants manufactured using 3D printing technologies boast increased functionality via 3D printing's inherent capability of building complicated surface textures and structures. However, while 3D printing provides improved capability for orthopedic implants, technical limitations of 3D printing prevent 3D printing from achieving complete separation between surface and structure complexities. By employing etching chemistries and technologies, the separation between surface and structure complexity is expanded, further improving the functional capabilities of the orthopedic implants.

Orthopedic implants are medical devices to treat musculoskeletal malformities or deficiencies. For example, these devices may have three primary components: a physical structure that replaces or supports the ailment; a surface modification for improved patient outcomes; and, internal fixation points to attach the device to the surrounding bone and/or soft tissues.

Advances and application of 3D printing towards the manufacturing of orthopedic implants has greatly improved their functionality by expanding on the potential complexity and customizability of these devices. Examples of this increased complexity and customizability include: introducing latticing to the physical structure, thereby modulating the mechanical properties of the implant to better match the surrounding skeletal components; expanding on surface roughness/porosity capabilities for improved osseointegration; and, enabling additional internal fixation points that would be too labor-intensive or difficult to manufacture using conventional machining.

Orthopedic implants are often made from titanium because of its non-toxicity, biocompatibility, and osseointegration capabilities. Furthermore, titanium readily forms a thin titanium oxide layer to act as a barrier, preventing adverse interaction with the underlying material. Advantageously, titanium powder is widely available and is compatible with most metal 3D printing technologies.

The present application provides chemical and physical material removal processes to modify the surface and/or structure of 3D-printed orthopedic implants. These implants improve upon the structural and lattice/porosity range introduced by the previous-generation implants while maintaining most, if not all, of the previous-generation's benefits. This has the potential to improve patient care and reduce costs related to implant complications.

Selective etching processes using precision and proven semiconductor-based etching processes on medical implants, particularly 3D printed medical implants, may also be used. These processes include but are not limited to wet etching for isotropic material removal and dry etching (plasma or reactive-ion) for anisotropic material removal. Selective etching is achieved by passivating or protecting desired locations with materials that are not etched by the process. Because semiconductor etchants are highly controlled processes that can range from tens of angstroms to tens of nanometers per second depending on etchant formulation/concentration and processing temperature, these procedures may be fine-tuned for precise material removal. Isotropic material removal is ideal for thinning struts and removing undesired surface roughness. Anisotropic material removal is ideal for increasing surface versatility while protecting the interior structure.

In a specific embodiment, the implants may be etched anisotropically using reactive ion etching or inductively coupled plasma using chemistries which are based on chlorine and fluorine containing gases. The reactive gases which may be used to etch titanium include but are not limited to CCl4/O2 based, Cl2/BCl3, Cl2/N2, Cl2/Ar, CF4, CF4/O2, SiCl4 and SF6. The gases as well as other process parameters may be chosen based on speed of etch and selectivity to the mask layer requirements.

For internal structure processing (isotropic etching), the medical implants may be designed with their final mechanical properties in mind such that the structures are dilated/expanded to account for the material removal to create the as-printed design. The as-printed design is printed and subsequently etched to remove the excess material to faithfully manufacture the as-designed implant. Procedure for designing an implant for anisotropic etching is similar to the isotropic procedure with the exception that dilation/expansion occurs uniaxially to account for the directional nature of dry etching.

A challenge relating to etching of medical implants, such as orthopedic implants, stems from their inherent three dimensional structure. Because semiconductors are essentially flat surfaces, application of passivation layers and etching is relatively trivial in traditional semiconductor processing. In the case of anisotropic etching of three dimensional medical implants, etching reactors may be designed to ensure that optimized etching properties may be targeted at the desired z-location (i.e., three dimensional feature location).

It is understood that several combinations of nano surface modulation may be used in parallel or in sequential order. Exemplary combinations include the use of optical shot peening followed by glass bead blasting to remove the burnt residuals that were generated by the laser. Another exemplary combination is the use of a non-selective traditional abrasive bead blasting to generate an initial homogeneous surface followed by selective nano surface modulation through electropolishing.

It is also understood that the combinations of a single method of nano surface modulation may be used to achieve selective nano surface modulation by varying the parameters of the surface modification and contact surfaces.

In some embodiments of the invention the selective nano surface modulation may be performed in a vacuum to prevent incorporation of contaminants such as oxides or organics that may occur under room air surface modulation.

In some embodiments of the invention, the selective nano surface modulation may be performed in a vacuum that is flooded with an inert gas such as argon.

It is understood that although the intended application of the selective nano surface modulation is to reduce irritation of neural, cartilage, or tendon structures from a medical device contact surface there may be other advantages such as modifying residual stresses to enhance damage tolerance or metal fatigue and reduce fretting wear and corrosion of the medical implant. In medical devices intended to terminate function, the modification may be performed to accelerate fatigue failure or corrosion.

Importantly, although limitation of contaminants may be an advantageous capability of the current invention to reduce irritation of neural, cartilaginous, or tendon structures from a medical device contact surface, there are scenarios where nitrides, oxides, or organics may provide beneficial nano surface modulation characteristics to a contact surface and a vacuum or inert gas is not used. For example, selective nano surface modulation in a nitrogen rich environment may lead to nitrides that are beneficial for bone growth and formation (i.e. titanium nitride). It is also understood that addition of contaminants during selective nano surface etching (i.e., silicon) may be used advantageously in the presence of a nitrogen rich environment for the generation of silicon nitride, which is beneficial for bone growth.

In some embodiments of the invention, several combinations environmental conditions are used in parallel or sequentially to apply selective nano surface modulation to different contact surfaces.

### EXAMPLES

Anterior lumbar interbody fusion is a commonly performed spine surgery in which a lumbar intervertebral disc is replaced by medical device called an “interbody cage.” In one embodiment of the invention, the interbody cage is in the general form of an ovoid cylinder manufacturing in Ti6Al4V-ELI through 3D printing. The portions of the cylinder in contact with the vertebral end plates may have a large 1 to 2 mm sawtooth pattern (i.e., 1 to 2 mm in height from tooth peak to between teeth valley) introduced during primary manufacturing to enhance friction of the interbody cage against the vertebral endplates. The posterior portion of the interbody cage is in close proximity or direct contact with neural structures, specifically the posterior longitudinal ligament of the spine, the dura which seals the cerebrospinal fluid around nerve rootlets or the spinal cord, and potentially exiting or traversing nerve roots. The contact surfaces on the posterior portion of the interbody cage selectively benefit from a smoother surface quality than the contact surfaces that are in contact or close proximity to the end plates. A selective nano surface modulation is achieved by applying a broad electropolishing process to provide an initial coarse surface finishing to all contact surfaces of the interbody cage. The anterior, superior, and inferior portions of interbody cage are then electrically insulated and the implant then undergoes a second more extensive electropolishing process to further refine the posterior portion of the interbody cage.

Shoulder resurfacing is a form of hemiarthroplasty where the articular surface of the humeral head is replaced with a prosthetic device that articulates with the native glenoid. In one embodiment of the invention, the shoulder resurfacing implant is in the general form of a hemispherical implant manufactured in Ti6Al4V-ELI through 3D printing. The back surface portion of the hemisphere in contact with the humerus may have keels or stems or other latticed structure introduced during primary manufacturing to provide a bone on-growth interface. The medial facing portion of the shoulder hemiarthroplasty is in close proximity or direct contact with the glenoid cartilage as well as tissues in close proximity which are capable of cascading contact with the glenoid cartilage such as the labrum, biceps tendon, and capsular tissue and intra-articular superior, medial and inferior glenohumeral ligaments. The contact surfaces on the medial hemispherical portion of the humeral head hemiarthroplasty implant selectively benefit from a smoother surface quality than the contact surfaces that are intended for contact with the remainder of the humerus. A selective nano surface modulation is achieved by applying a broad electropolishing process to provide an initial coarse surface finishing to all contact surfaces of the hemiarthroplasty device. The contact surfaces on the medial facing portion of the hemiarthroplasty is then selectively exposed to a second more extensive electropolishing process to further refine the glenoid contacting portion of the implant with the humeral contacting portions unexposed to electrolyte.

Distal radius fractures are commonly treated using volar plates. Tendon rupture is a concern with these medical devices as the flexor tendons cross the plate and heads of the plate screws during normal physiologic wrist and finger motion. In one embodiment of the invention, the distal radius plate is in the general form of a T-shaped plate manufactured in Ti6Al4V-ELI through 3D printing. The back surface portion of the plate in contact with the bone may have scallops or a latticed structure introduced during primary manufacturing to provide a bone on-growth or in-growth interface. The volar facing portion of the plate is in close proximity or direct contact with the flexor tendons of the hand and wrist as well as tissues in close proximity which are capable of cascading contact with tendons such as the pronator quadratus and flexor carpi muscles. The contact surfaces on the volar portion of the plate and heads of the screws selectively benefit from a smoother surface quality than the contact surfaces that are intended for contact with bone. A selective nano surface modulation is achieved by applying an electropolishing process on the volar surface of the plate and heads of the screws while insulating the bone contact surface of the plate and threads of the screws unexposed to electrolyte.

In another example, the previously described interbody cage undergoes a single stage electropolishing process where only the posterior portion of the interbody cage is contact with the electrolyte to achieve a selective nano surface modulation.

In another example, the previously described hemiarthroplasty undergoes a single stage dry plasma etching process where the portions of the hemiarthroplasty intended for bone in-growth or on-growth are masked, and the remaining contact surfaces are etched in a dry plasma etch to achieve a selective nano surface modulation.

In another example, the previously described volar plate and screw set undergo a single stage dry plasma etching process where the portions of the implant intended for bone in-growth or on-growth are masked, and the remaining contact surfaces are etched in a dry plasma etch to achieve a selective nano surface modulation of the surfaces in contact or in close proximity to tendons.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

The foregoing description of the exemplary embodiments is provided to enable any person skilled in the art to make or use the claimed subject matter. Various modifications to these embodiments will be readily apparent to those skilled in the art, and the generic principles defined herein may be applied to other embodiments without the use of the innovative faculty. Thus, the claimed subject matter is not intended to be limited to the embodiments shown herein but is to be accorded the widest scope consistent with the principles and novel features disclosed herein.

What is claimed is:

1. A method for forming a medical device, the method comprising:
forming a three dimensional medical structure, said three dimensional structure having x, y, and z structural geometry in a three dimensional x, y, and z Cartesian coordinate system; and,
selectively modulating surface roughness of a portion of said three dimensional medical structure to have surface roughness features less than 0.1 mm.

2. The method of claim 1, wherein said step of selectively modulating surface roughness of a portion of said three dimensional medical structure is performed using a low plasticity burnishing process.

3. The method of claim 1, wherein said step of selectively modulating surface roughness of a portion of said three dimensional medical structure is performed using an electropolishing process.

4. The method of claim 1, wherein said step of selectively modulating surface roughness of a portion of said three dimensional medical structure is performed using an optical peening process.

5. The method of claim 1, wherein said step of selectively modulating surface roughness of a portion of said three dimensional medical structure is performed using an ultrasonic shot peening process.

6. The method of claim 1, wherein said step of selectively modulating surface roughness of a portion of said three dimensional medical structure is performed using a dry etching process.

7. The method of claim 6, wherein said dry etching process is a reactive ion etching process.

8. The method of claim 6, wherein said dry etching process is an inductively coupled plasma etching process.

9. The method of claim 1, wherein said step of selectively modulating surface roughness of a portion of said three dimensional medical structure is performed using a wet etching process.

10. The method of claim 1, wherein said step of selectively modulating surface roughness of a portion of said three dimensional medical structure is performed using an optical shot peening process and a glass bead blasting process.

11. The method of claim 1, wherein said step of selectively modulating surface roughness of a portion of said three dimensional medical structure is performed using an abrasive bead blasting process and an electropolishing process.

12. A medical device, comprising:
a three dimensional medical structural component having x, y, and z structural geometry in a three dimensional x, y, and z Cartesian coordinate system, said three dimensional structural component made of at least a structural material and a nano-modulated surface roughness on a portion of the implant that includes at least one but not all contact surfaces; the nano-modulated surface roughness controlled via the addition, coating, deposition, or deformation of structural or non-structural material in valleys of the contact surface; the nano-modulated surface roughness controlled via the removal, coating, deposition, or deformation of material in the peaks of the contact surface; and,
the surface roughness of a portion of said three dimensional medical structure to have surface roughness features less than 0.1 mm.

13. The medical device of claim 12, wherein said nano-modulated surface roughness is controlled via a low plasticity burnishing process.

14. The medical device of claim 12, wherein said nano-modulated surface roughness is controlled via an electropolishing process.

15. The medical device of claim 12, wherein said nano-modulated surface roughness is controlled via an optical peening process.

16. The medical device of claim 12, wherein said nano-modulated surface roughness is controlled via an ultrasonic shot peening process.

17. The medical device of claim 12, wherein said nano-modulated surface roughness is controlled via a dry etching process.

18. The medical device of claim 17, wherein said dry etching process is a reactive ion etching process.

19. The medical device of claim 17, wherein said dry etching process is an inductively coupled plasma etching process.

20. The medical device of claim 12, wherein said nano-modulated surface roughness is controlled via a wet etching process.
